(19) United States
(12) Patent Application Publication
(10) Pub. No.: US 2023/0392110 A1
(43) Pub. Date: Dec. 7, 2023

(54) ANTAGONIST MICROORGANISMS FOR INHIBITING FIRE BLIGHT AND COMPOSITION FOR INHIBITING FIRE BLIGHT WITH THE SAME AS ACTIVE INGREDIENT

(71) Applicant: KNU-INDUSTRY COOPERATION FOUNDATION, Chuncheon-si (KR)

(72) Inventors: Duck Hwan PARK, Chuncheon-si (KR); Dong Hyuk CHOI, Chuncheon-si (KR); Hyun Ju CHOI, Chuncheon-si (KR); Yeon Ju KIM, Seoul (KR); Yeon Jeong LIM, Jeonju-si (KR); In Gyeong LEE, Seoul (KR)

(73) Assignee: KNU-INDUSTRY COOPERATION FOUNDATION, Chuncheon-si (KR)

(21) Appl. No.: 18/328,308

(22) Filed: Jun. 2, 2023

(30) Foreign Application Priority Data
Jun. 3, 2022 (KR) 10-2022-068109
Feb. 8, 2023 (KR) 10-2023-0016570

(51) Int. Cl. C12N 1/20; A01N 63/22; A01P 1/00

(52) U.S. Cl. CPC C12N 1/20; A01N 63/22; A01P 1/00

(57) ABSTRACT

The present invention relates to antagonist microorganisms for inhibiting fire blight and a composition for preventing or inhibiting fire blight comprising the same as an active ingredient, and provides antagonist microorganisms exhibiting excellent antagonistic ability against a causative bacterium, Erwinia amylovora. According to the present invention, the antagonist microorganisms for inhibiting fire blight isolated in Korea are provided to replace a biological control agent for inhibiting fire blight, which requires enormous costs depending on imports.

### CROSS-REFERENCE TO RELATED APPLICATIONS

This application is based on and claims priority from Korean Patent Application No. KR10-2022-0068109, filed on Jun. 3, 2022, and Korean Patent Application No. KR10-2023-0016570, filed on Feb. 8, 2023, with the Korean Intellectual Property Office, the disclosure of which is incorporated herein in its entirety by reference.

### TECHNICAL FIELD

The present disclosure relates to antagonist microorganisms for inhibiting fire blight and a composition for inhibiting fire blight comprising the antagonist microorganisms as an active ingredient.

### BACKGROUND

Fire blight is a bacterial disease that affects some plants, such as apples, pears and rosaceae, and the causative pathogen is Erwinia amylovora. The fire blight mainly occurs during a flowering period and is transfected by the motility of bees or pathogens washed by rain. When infected with the fire blight, tissues such as leaves, flowers, branches, stems, and fruits turn black and gradually wither and die. There is no treatment or control drug, and the speed of spread is high, so that the scale of damage is large.

Since the fire blight was first reported in the eastern United States in 1780, the fire blight has occurred in many countries including North America and Europe. In Korea, after the first outbreak in Anseong, Gyeonggi-do in 2015, new outbreaks occurred in Cheonan and Anseong in 2017, Chungju, Jecheon, Wonju, and Pyeongchang in 2018, and Paju, Yeoncheon, Icheon, and Yongin in 2019, and the area of occurrence is gradually spreading. In particular, in the Jecheon and Chungju regions, economic damage was severe due to the closure of apple and pear orchards according to large-scale outbreaks, and in addition to the affected area, there is a possibility of spreading to major fruit complexes such as Yeongju, Bonghwa, and Yecheon in Gyeongbuk, which are the main apple producing areas, and Danyang, Cheongju, and Okcheon in Chungbuk, and thus, there is a need for an alternative to block the new occurrence of fire blight and prevent its spread.

Proliferation prevention policies are conducted according to each region divided into occurring regions, buffer regions, and non-occurring regions of the fire blight, and attempts are being made to block new outbreaks and prevent the spread of the fire blight by treatment of control agents, but in environment-friendly cultivation orchards, it is not possible to process chemicals, and in many cases, organic materials for suppressing other diseases or self-developed unregistered treatment agents are used.

In order to solve this problem, the present disclosure is to develop and provide a biological control agent that can be used in orchards nationwide.

### SUMMARY

The present disclosure has been made in an effort to provide novel antagonist microorganisms for inhibiting Erwinia amylovora, a causative bacterium of fire blight, and a composition for inhibiting fire blight including the same.

An exemplary embodiment of the present disclosure provides a Bacillus altitudinis KPB25 (Accession Number: KACC81238BP) strain.

In the present disclosure, the strain may have antagonistic ability against preferably Erwinia amylovora strain, but is not limited thereto.

Another exemplary embodiment of the present disclosure provides a Bacillus safensis KPB31 (accession number: KACC81239BP) strain.

In the present disclosure, the strain may have antagonistic ability against preferably Erwinia amylovora strain, but is not limited thereto.

Yet another exemplary embodiment of the present disclosure provides a composition for preventing or inhibiting fire blight comprising at least one strain selected from a Bacillus altitudinis KPB25 (Accession Number: KACC81238BP) strain; or a Bacillus safensis KPB31 (Accession Number: KACC81239BP) strain as an active ingredient.

In the present disclosure, the causative bacterium of the fire blight may be Erwinia amylovora, but is not limited thereto.

In the present disclosure, the fire blight may be caused by preferably apples, but is not limited thereto.

Still another exemplary embodiment of the present disclosure provides a biological control agent including the composition for preventing or inhibiting fire blight.

Still yet another exemplary embodiment of the present disclosure provides a method for preventing or inhibiting fire blight, including treating a plant with the composition for preventing or inhibiting fire blight.

In the present disclosure, the plant may be preferably apples, but is not limited thereto.

According to the present disclosure, antagonist microorganisms for inhibiting fire blight isolated in Korea are provided to replace a biological control agent for inhibiting fire blight, which requires enormous costs depending on imports

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating isolation sources of 45 types of microorganisms according to an example of the present disclosure.

FIG. 2 is a diagram illustrating strains exhibiting antagonistic activity against Erwinia amylovora among 45 types of microorganisms according to an example of the present disclosure.

FIG. 3 is a diagram for identifying the antagonistic activity against Erwinia amylovora and sizes of inhibition zones of five strains KPB15, 21, 25, 31, and 39 according to an example of the present disclosure.

FIG. 4 is a diagram of confirming differences in activity of five strains KPB15, 21, 25, 31, and 39 depending on medium conditions according to an example of the present disclosure.

FIG. 5 is a diagram of confirming motilities of five strains KPB15, 21, 25, 31, and 39 according to an example of the present disclosure.

FIG. 6 is a diagram of confirming the antagonistic ability against Erwinia amylovora of five strains KPB15, 21, 25, 31, and 39 in immature apples according to an example of the present disclosure.

FIG. 7 is a diagram of analyzing sizes of necrosis lesions observed in appearance of immature apples by Image J according to an example of the present disclosure.

FIG. 8 is a diagram of confirming the antagonistic ability against Erwinia amylovora of five strains KPB15, 21, 25, 31, and 39 in apple seedlings according to an example of the present invention.

FIG. 9 is a diagram showing the disease severity observed in the appearance of apple seedlings according to an example of the present invention.

FIG. 10 is a diagram of confirming the density of fire blight causative bacteria surviving in apple seedlings according to an example of the present invention.

FIG. 11 is a diagram of a phylogenetic tree of KPB15 according to an example of the present invention.

FIG. 12 is a diagram of a phylogenetic tree of KPB21 according to an example of the present invention.

FIG. 13 is a diagram of a phylogenetic tree of KPB25 according to an example of the present invention.

FIG. 14 is a diagram of a phylogenetic tree of KPB31 according to an example of the present invention.

FIG. 15 is a diagram of a phylogenetic tree of KPB39 according to an example of the present invention.

FIG. 16 is a diagram of confirming the antagonistic ability between five strains KPB15, 21, 25, 31, and 39 according to an example of the present invention.

FIG. 17 is a diagram showing an inhibition zone assay of enhanced H₂O₂ tolerance strains KPB25-HP and KPB31-HP according to an example of the present invention.

FIG. 18 is a diagram showing an inhibition zone assay result of enhanced H₂O₂ tolerance strains KPB25-HP and KPB31-HP according to an example of the present invention.

FIG. 19 is a diagram of confirming inhibition of Erwinia amylovora by KPB25-HP through genetic analysis according to an example of the present invention.

FIG. 20 is a diagram of confirming additional usable substances of KPB25-HP and KPB31-HP according to an example of the present invention.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in more detail through Examples and Experimental Examples. However, the following Examples and Experimental Examples are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

### <Example 1> Isolation of Antagonist Microorganisms for Direct Inhibition of Erwinia amylovora

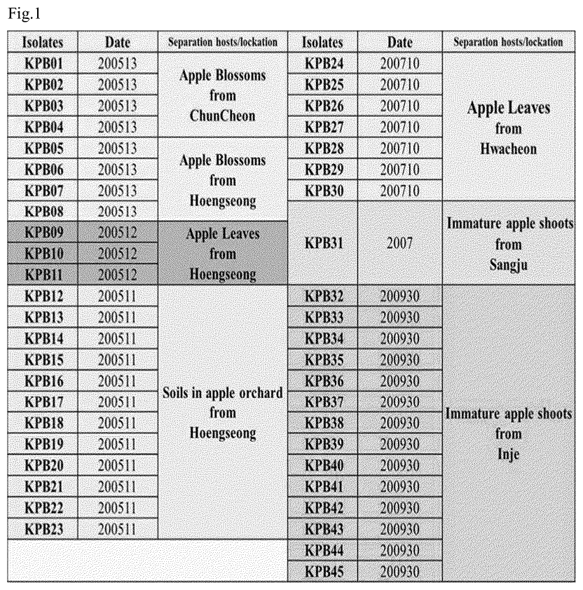

A total of 45 microorganisms were isolated from apple blossoms, apple shoots or soils in apple orchard (FIG. 1). The isolated microorganisms were named Kangwon National University Plant Bacteria (KPB) in order. In order to confirm a direct inhibitory effect of the isolated microorganism on Erwinia amylovora, an inhibition zone (clear zone) assay was performed.

An Erwinia amylovora TS 3128 strain was diluted in a 10 mM MgCl₂ buffer to a concentration of OD_{600 nm}=0.1 and smeared on an MGY medium, and then the 45 microorganisms were diluted at the same concentration and inoculated in an amount of 10 μL to identify the antagonistic degree of showed inhibition zones.

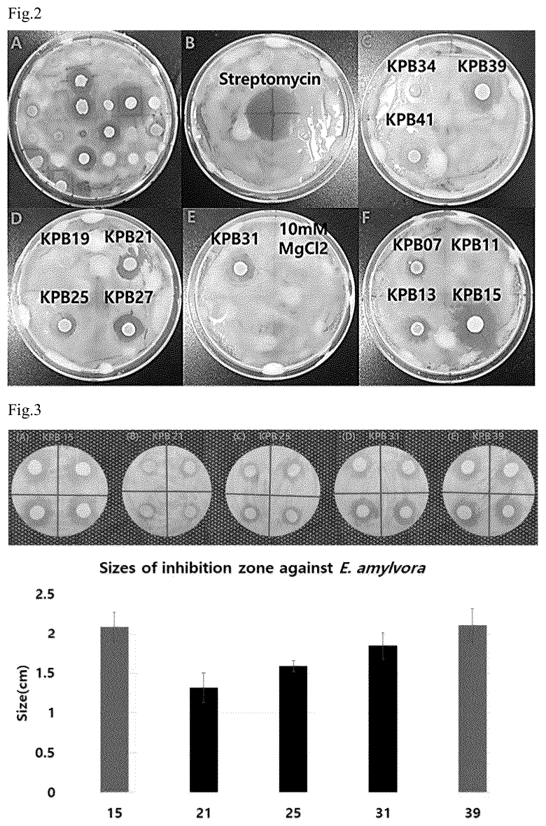

Among the 45 types of microorganisms, strains exhibiting excellent antagonistic ability against Erwinia amylovora were shown in FIG. 2A. FIG. 2B is a diagram confirming the formation of inhibition zones according to the treatment with streptomycin as an antibiotic. As shown in FIGS. 2C to 2F, five strains KPB15, 21, 25, 31, and 39 with the clearest inhibition zones were selected.

The five strains KPB15, 21, 25, 31, and 39 were treated in the same manner as in the method to confirm the formation of inhibition zones. As shown in FIG. 3, the inhibition zones of 1 cm or more were confirmed in all of the five strains KPB15, 21, 25, 31, and 39.

### <Example 2> Confirmation of Activity Depending on Medium Conditions

To confirm activity depending on medium conditions, the antagonistic ability against Erwinia amylovora of five strains KPB15, 21, 25, 31, and 39 was confirmed in Erwinia amylovora semi-selective media KB, MGY, LB, NAG, R2A, and TSA (Artur Mikicinski et al. 2020).

An Erwinia amylovora TS 3128 strain and five strains KPB15, 21, 25, 31, and 39 were incubated on an LB solid medium for 18 hours, and then each colony was suspended in a 10 mM MgCl₂ buffer at a concentration of OD_{600 nm}=0.1. 100 μL of the Erwinia amylovora TS 3128 strain suspension was smeared on KB, MGY, LB, NAG, R2A, and TSA media, respectively, and after the smeared portions were all dry, 20 μL of the suspension of the five strains KPB15, 21, 25, 31, and 39 was inoculated and incubated for 24 hours.

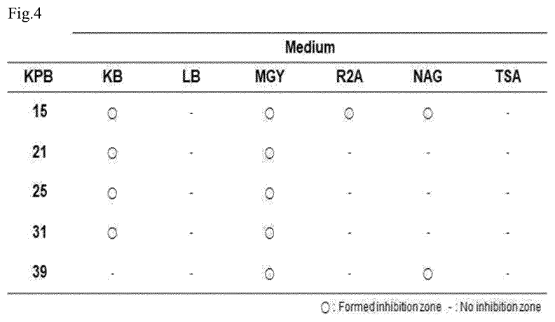

FIG. 4 is a diagram illustrating results of confirming whether an inhibition zone is formed depending on each medium condition. In an MGY medium, all of the five strains KPB15, 21, 25, 31, and 39 formed inhibition zones, and in a KB medium, four strains except for KPB39 strain formed inhibition zones. Meanwhile, the strains KPB15 and 39 formed inhibition zones in a NAG medium.

### <Example 3> Confirmation of Motility of Antagonist Microorganisms

The motility of microorganisms on a plant surface may affect the antagonistic ability. According to preliminary experiments, the motility of five strains KPB15, 21, 25, 31, and 39 was confirmed using a KB medium with the highest motility.

Erwinia amylovora TS 3128km^{R} (pBAV1K: kanamycin resistant plasmid) and the five strains KPB15, 21, 25, 31, and 39 were incubated in a KB medium for 18 hours, and then each colony was suspended in a 10 mM MgCl₂ buffer at a concentration of OD_{600 nm}=0.1. 100 μL of the Erwinia amylovora TS 3128km^{R} dilution was smeared, and 10 μL of the suspension of the five strains KPB15, 21, 25, 31, and 39 was inoculated in the center of the medium, and then incubated for 48 hours. As a control, only 100 μL of the Erwinia amylovora TS 3128kmR suspension was smeared and incubated for 48 hours. A sample was taken, mixed with 1 mL of an MgCl₂ buffer, pulverized, and sufficiently vortexed. Thereafter, the mixture was 10-fold diluted at concentrations of 10⁻¹ to 10⁻⁷, spot-inoculated and incubated in a KB/Km medium for 24 hours, and then the CFU was measured.

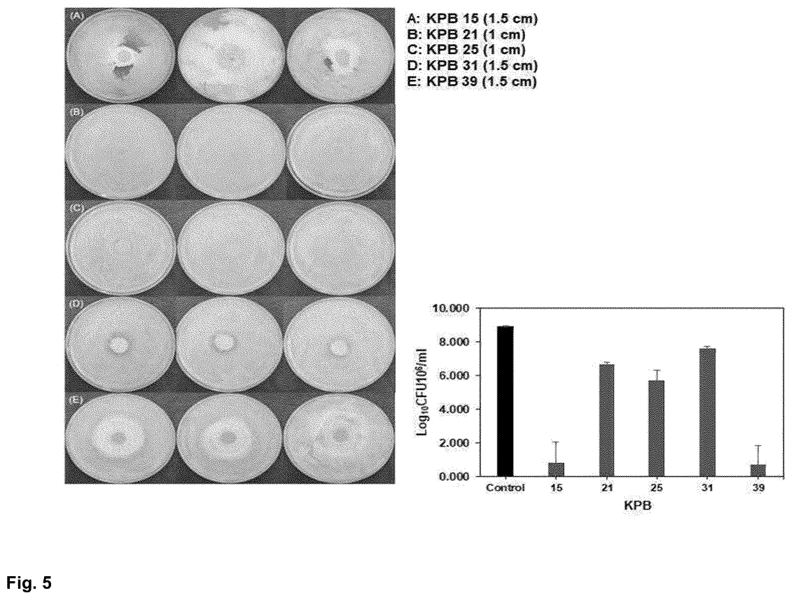

As shown in FIG. 5, it was confirmed that the motility was high in KPB15 and 39, and an inhibition zone was formed in KPB31.

### <Example 4> Confirmation of Effect of Inhibiting Fire Blight in Immature Apples

In this experiment, it was confirmed whether five strains KPB15, 21, 25, 31, and 39 inhibited fire blight in immature apples (cv. Fuji). As confirmed in Example 2, holes were bored into the surfaces of the immature apples using a cork borer. KPB15 and 39 with high motility and KPB31 with an inhibition zone were bored at 1.5 cm from the surfaces of immature apples to confirm the antagonistic ability and the remaining KPB21 and 25 strains was bored at 1 cm from the surfaces of immature apples to confirm the antagonistic ability.

The surfaces of immature apples were sterilized with a 2% sodium hypochlorite solution and washed three times in a cycloheximide solution (1000×) as a protein biosynthesis inhibitor in eukaryotes. 4-mm holes were bored into the surfaces of the sterilized immature apples, and immersed for 20 minutes in a suspension of each of the five strains KPB15, 21, 25, 31, and 39 diluted at a concentration of OD_{600 nm}=0.1 in a 10 mM MgCl₂ buffer. Thereafter, 10 μL of the Erwinia amylovora TS 3128 strain suspended at the same concentration was inoculated into the holes, and then incubated at 28° C. for 7 days to induce disease symptom development. A control group was inoculated with only the Erwinia amylovora TS 3128 strain.

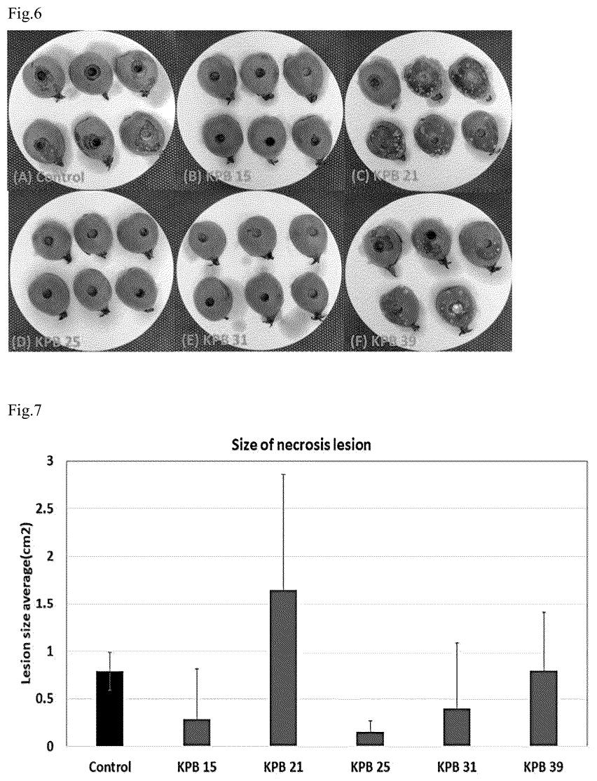

FIG. 6 is a diagram of observing the appearance of immature fruits at 7 days after inoculation. The diameter of the necrotic area was regarded as a lesion, and the necrotic area of the observed symptom was analyzed using Image J. As shown in FIG. 7, all antagonist microorganisms except the KPB21 strain inhibited the Erwinia amylovora TS 3128 strain, and in particular, KPB15, 25 and 31 strains effectively inhibited the Erwinia amylovora TS 3128 strain.

### <Example 5> Confirmation of Effect of Inhibiting Fire Blight in Apple Seedlings

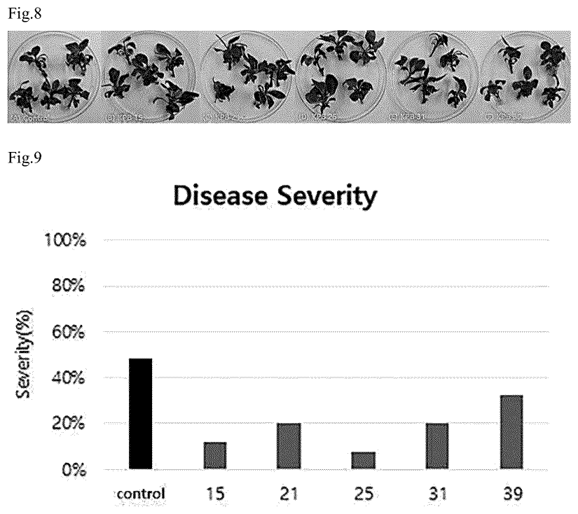

The antagonistic ability was tested using apple (cv. M9) seedlings in the same manner as in Example 2, and the surface sterilization process was omitted. Erwinia amylovora TS 3128^{R} (pBAV1K: kanamycin resistance plasmid) was used, and inoculated and then stored in a constant temperature room at 28° C. for 10 days to induce the onset of disease. The disease severity was classified as shown in Table 1 below according to the number of necrotized leaves or leafstalks. FIG. 8 is a diagram of observing the appearance of apple seedlings at 10 days after inoculation.

TABLE 1

| scale | Disease severity |
|---|---|
| 0 | No symptom |
| 1 | 1 necrotized leaf or leafstalk |
| 2 | 2 necrotized leaves or leafstalks |
| 3 | 3 necrotized leaves or leafstalks |
| 4 | 4 necrotized leaves or leafstalks |
| 5 | At least 5 necrotized leaves or leafstalks |

As shown in FIG. 9, it was confirmed that compared to an untreated group showing the disease severity of 40% or more, the five strains KPB15, 21, 25, 31, and 39 of the present invention exhibited the disease severity of 10% to 30% to have a fire blight inhibitory effect.

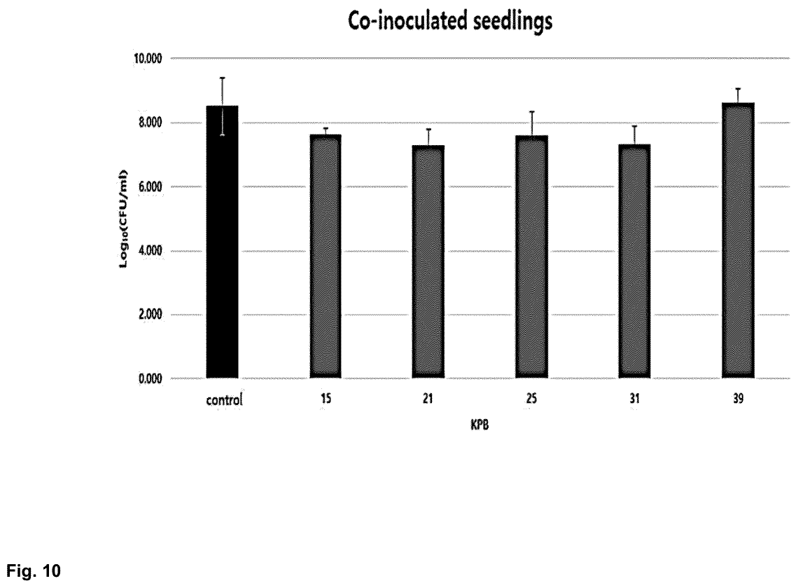

In addition, as shown in FIG. 10, as a result of confirming the density (CFU/mL) of the Erwinia amylovora TS 3128^{R} (pBAV1K: kanamycin resistant plasmid) strain surviving on apple seedlings at 10 days after inoculation, the proliferation inhibitory effect of Erwinia amylovora TS 3128^{R} (pBAV1K: kanamycin resistant plasmid) was confirmed in the remaining four strains KPB15, 21, 25, and 31 except for the KPB39 strain. Accordingly, it was determined that the five strains KPB15, 21, 25, 31, and 39 of the present invention inhibited the proliferation of Erwinia amylovora in a host plant below a specific density required for disease symptom development.

### <Example 6> Identification of Five Strains KPB15, 21, 25, 31, and 39

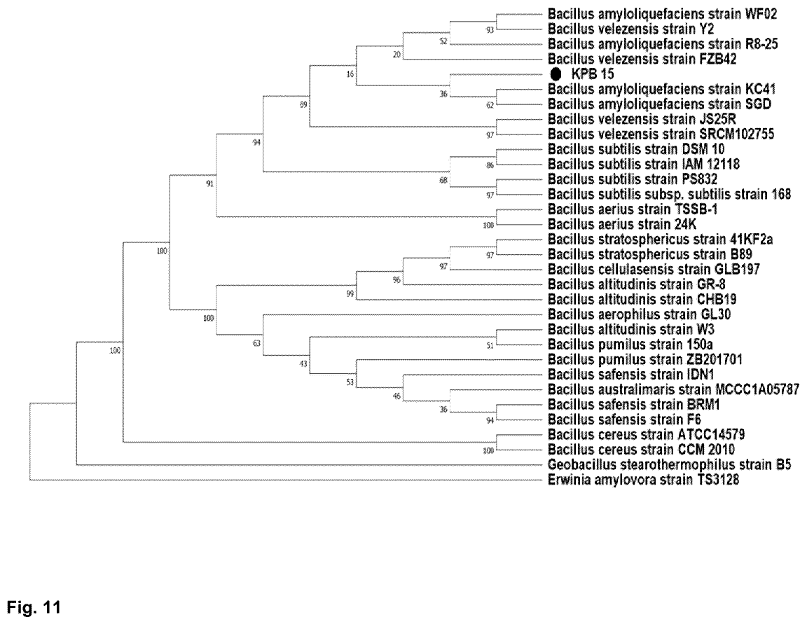

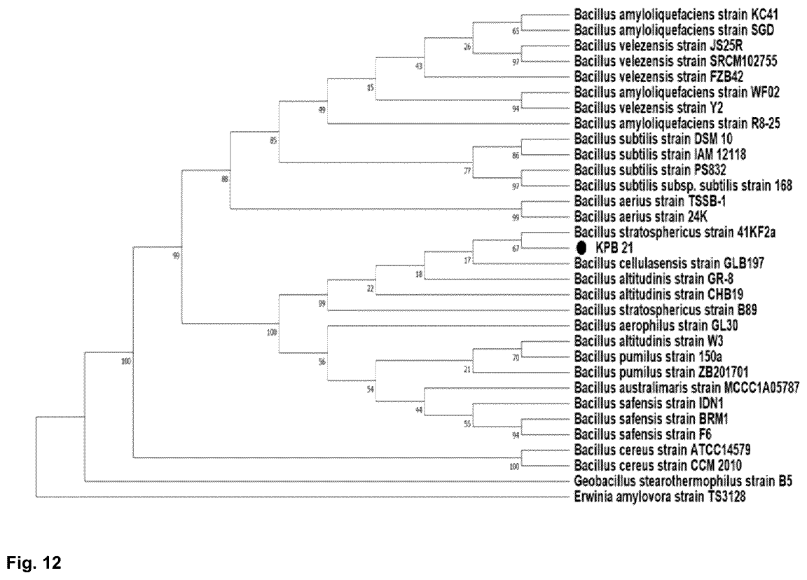

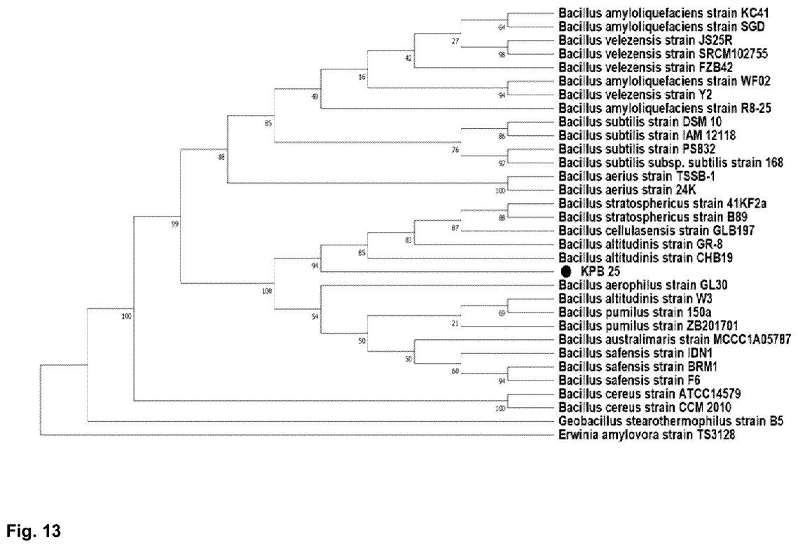

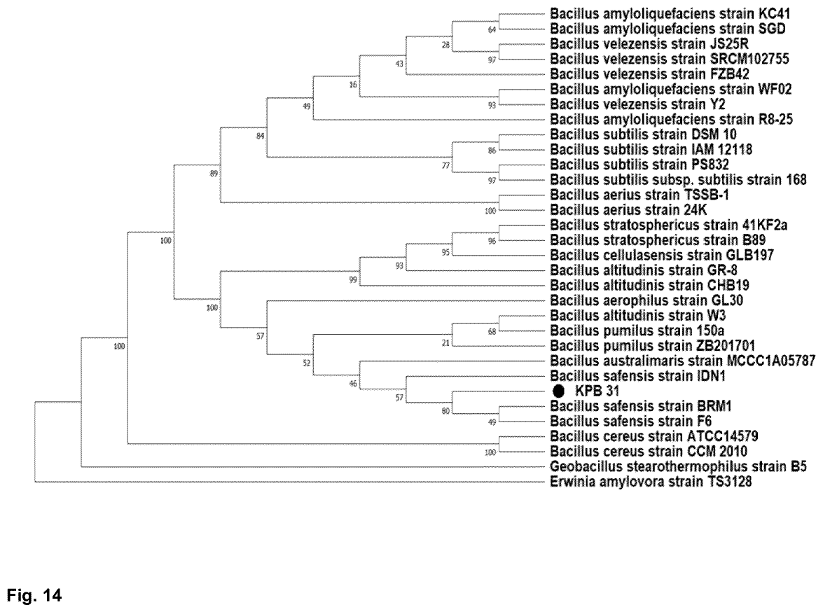

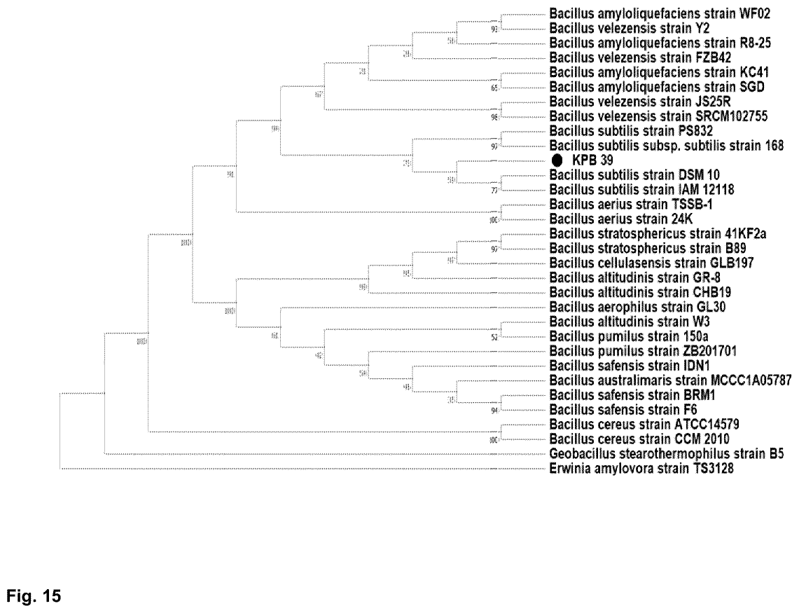

Five strains KPB15, 21, 25, 31, and 39 were identified based on 16S rRNA sequences. A 16S rRNA region amplified from genomic DNA was cloned using an INVITROGEN TOPO® TA Cloning® Kit and sequenced, and the 16s rRNA gene sequences of the analyzed representative strains were confirmed by a Basic Local Alignment Search Tool (BLAST) in GeneBank International Database of the National Center for Biotechnology Information (NCBI). A phylogenetic tree using the BLASTed 16S rRNA region sequence was constructed using a MEGA X program, and the phylogenetic tree was constructed through a Neighbor-joining method by performing alignment with a ClustalW algorithm (around Bootstrap replications 500). FIG. 11 showed a phylogenetic tree of KPB15, FIG. 12 showed a phylogenetic tree of KPB21, FIG. 13 showed a phylogenetic tree of KPB25, FIG. 14 showed a phylogenetic tree of KPB31, and FIG. 15 showed a phylogenetic tree of KPB39.

TABLE 2

| KPB | Identified species |
|---|---|
| 15 | Bacillus amyloliquefaciens |
| 21 | Bacillus stratosphericus |
| 25 | Bacillus altitudinis |
| 31 | Bacillus safensis |
| 39 | Bacillus subtilis |

All five strains were identified as belonging to the genus Bacillus, and specific species were identified as shown in Table 2 above. KPB15 (Bacillus amyloliquefaciens) and KPB39 (Bacillus subtilis) were species known as a fire blight causative bacterium (Erwinia amylovora) inhibitor, whereas KPB21 (Bacillus stratosphericus), KPB25 (Bacillus altitudinis), and KPB31 (Bacillus safensis) were identified as novel species previously unknown as fire blight causative bacterium (Erwinia amylovora) inhibitors.

### <Example 7> Identification of Coexistence of Antagonist Microorganisms

Five strains KPB15, 21, 25, 31, and 39 were suspended in a 10 mM MgCl₂ buffer at a concentration of OD_{600 nm}=0.1, respectively, and smeared on a KB medium, and then the remaining strains except for the smeared strains were inoculated with 10 μL and incubated for 24 hours.

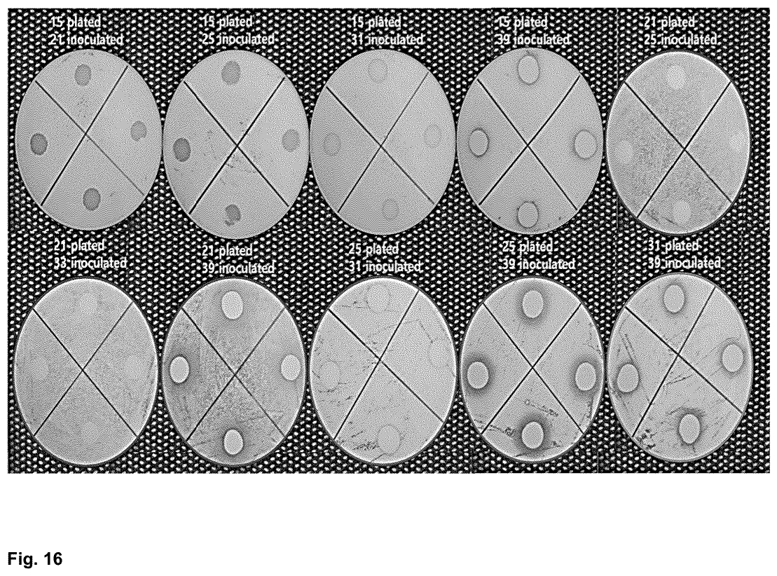

As shown in FIG. 16, the KPB21, 25 and KPB31 strains were confirmed to have antagonistic ability with the KPB39 strain, respectively, and confirmed to hardly coexist. Therefore, it was confirmed that the treatment was possible using the KPB25 and KPB31 strains of the present invention, which had excellent effects.

### <Example 8> Construction of Enhanced Oxidative Stress Tolerance Strains

Subsequently, it was targeted to develop an effective biological control agent by enhancing the characteristics of useful microorganisms KPB25 and KPB31 strains having inhibitory ability against Erwinia amylovora. The biological control agent was less effective in control effect in the field than chemical control agents, and the cause thereof was considered to be caused by various environmental stresses such as UV stress, osmotic stress, and oxidative stress. Of these, strains overcoming the oxidative stress were intended to be constructed (KBP15, 21, and 39 were excluded from the construction of enhanced oxidative stress tolerance strains because the strains could not withstand oxidative stress in this experiment).

First, in order to determine the H₂O₂ MIC of useful microorganisms, each strain of useful microorganisms was added with a H₂O₂ working solution (1 M H₂O₂) so as to adjust OD_{600 nm} to 0.01 density and to be at a desired molar concentration in an LB liquid medium. Thereafter the strains were incubated in a 28° C. shaking incubator, the Optical Density (OD) values were measured every 24 hours until 72 hours, and at this time, the concentration at which the incubation was completely inhibited by 72 hours was set as MIC (Table 3).

TABLE 3

| Strains | MIC of H₂O₂ |
|---|---|
| KPB25 | 5 mM |
| KPB31 | 5 mM |

Enhanced H₂O₂ tolerance strains were constructed by spontaneous mutagenesis, and each strain was incubated in an LB liquid medium containing 4 mM H₂O₂, which was a concentration below the MIC. The incubated strains were serially cultivated by inoculating 1/100 of the LB liquid medium containing a higher concentration of H₂O₂. Enhanced H₂O₂ tolerance strains capable of culturing at a final concentration of 20 mM H₂O₂ were constructed.

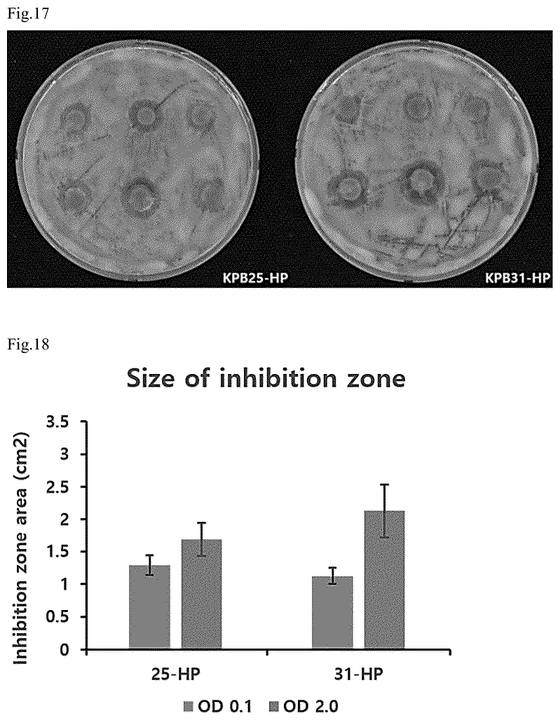

For an in vitro inhibition zone assay, the antagonistic ability against E. amylovora of 20 mM enhanced H₂O₂ tolerance strains KPB25-HP and KPB31-HP was confirmed through the inhibition zone assay. 100 μl of an E. amylovora suspension with OD_{600 nm}=0.1 density was smeared on an MGY solid medium, and antagonistic ability was confirmed by the degree of inhibition zone shown by tooth-inoculating 10 μl of each enhanced tolerance strain at the density of OD_{600 nm}=0.1 (top) and OD_{600 nm}=2.0 (bottom) (FIG. 17). In addition, as a result of quantification using the image J software program, all of the strains showed inhibition zones with sizes of 1 cm² or more (FIG. 18). As a result, enhanced oxidative stress tolerance strains that had 20 mM H₂O₂ tolerance and formed inhibition zones were constructed.

### <Example 9> Confirmation of Inhibition of Erwinia amylovora of KPB25-HP Through Genetic Analysis

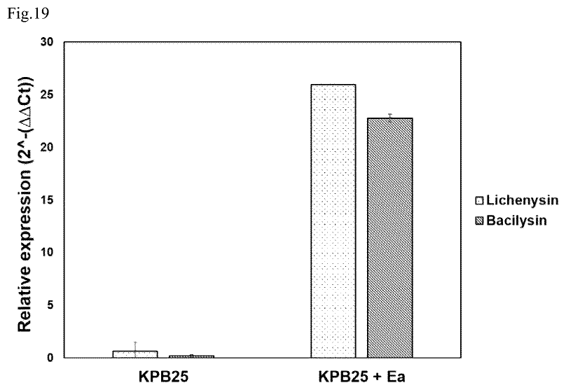

The genomic DNA of KPB25HP of the present invention was subjected to whole gene analysis by Pacbio sequencing. Among the gene regions analyzed, a coding sequence (CDS) of an antibacterial substance capable of inhibiting Erwinia amylovora was identified, and two of the most representative antibacterial substances Lichenysin and Bacilysin were selected and the following experiment was conducted to measure the expression levels thereof against Erwinia amylovora. 20 μl of KPB25-HP set to optical density (OD) of 0.1 nm was inoculated on MgCl₂ in a MGY liquid medium, and shaking-cultured for 24 hours after simultaneously inoculating Erwinia amylovora in the same concentration and amount as the useful microorganisms inoculated into an experimental group. The expression level of total RNA extracted from the cultured medium was confirmed on the medium by qRT-PCR using a SYBR reagent. As a result, as may be seen in FIG. 19, the expression levels of the two antibacterial substances shown in the medium inoculated with KPB25-HP alone and the expression level shown in the medium additionally inoculated with Erwinia amylovora were confirmed. When additionally inoculated with Erwinia amylovora, it was confirmed that the expression levels of antibacterial substances Lichenysin and Bacilysin were relatively increased. Therefore, it was confirmed that KPB25-HP used the corresponding antibacterial substances to inhibit Erwinia amylovora.

### <Example 10> Confirmation of Additional Usable Substances of KPB25-HP and KPB31-HP

The genomic DNA of KPB25HP and KPB31-HP was subjected to whole gene analysis by Pacbio sequencing. Total three categories: antibacterial substances used in other references, substances related to the plant induction resistance, and substances helping in the growth of the plant were divided and listed, and in the gene regions of the analyzed KPB25-HP and KPB31-HP, the coding sequences (CDS) of the corresponding substances were additionally confirmed to construct a heat map.

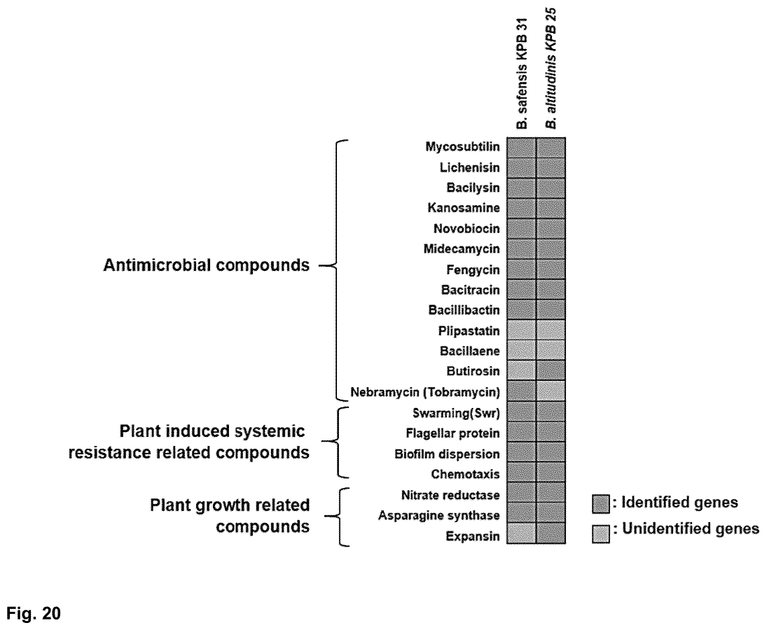

As may be seen in FIG. 20, in KPB25-HP and KPB31-HP, additional antifungal (Mycosubtilin) and antibacterial substances, plant systemic resistance-related substances and plant growth-related substances were also identified. Accordingly, KPB25-HP and KPB31-HP are expected to be used in various ways as useful microorganisms as well as inhibiting Erwinia amylovora.

As a result, according to Examples 1 to 10 above, Bacillus altitudinis KPB25-HP (Bacillus altitudinis KPB25; accession number: KACC81238BP) and Bacillus safensis KPB31-HP (Bacillus safensis KPB31; accession number: KACC81239BP) strains of the present invention were deposited at the Korean Agricultural Culture Collection (KACC) as strains with the best effect and enhanced oxidative stress tolerance, and the present invention was completed.

[Accession Number]
Depositary Authority Name: Korean Agricultural Culture Collection (KACC)
Accession number: KACC81238BP
Accession Date: 2022 Nov. 23
Depositary Authority Name: Korean Agricultural Culture Collection (KACC)
Accession number: KACC81239BP
Accession Date: 2022 Nov. 23

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A Bacillus altitudinis KPB25-HP (accession number: KACC81238BP) strain or Bacillus safensis KPB31-HP (accession number: KACC81239BP) strain, which is a novel strain with enhanced oxidative stress tolerance.

2. The strain of claim 1, wherein the strain has antagonistic ability to an Erwinia amylovora strain.

3. A composition for preventing or inhibiting fire blight, comprising the novel strain of claim 1 as an active ingredient.

4. The composition of claim 3, wherein the causative bacterium of the fire blight is Erwinia amylovora.

5. The composition of claim 4, wherein the fire blight is caused by apples.

6. A biological control agent comprising the composition for preventing or inhibiting fire blight of claim 3.

7. A method for preventing or inhibiting fire blight, comprising treating a plant with the composition for preventing or inhibiting fire blight of claim 3.

8. The method of claim 7, wherein the plant is apples.
